(19) United States
(12) Patent Application Publication
(10) Pub. No.: US 2013/0334131 A1
(43) Pub. Date: Dec. 19, 2013

(54) IDENTIFICATION OF AND COMPOSITIONS CONTAINING POLYPHOSPHATE ACCUMULATING BACTERIA

(71) Applicants: UNIVERSITY OF NORTH TEXAS, Denton, TX (US); TENFOLD TECHNOLOGIES LLC, Pilot Point, TX (US)

(72) Inventors: Michael Allen, Denton, TX (US); Michael LaMontagne, Denton, TX (US)

(21) Appl. No.: 13/842,400

(22) Filed: Mar. 15, 2013

Related U.S. Application Data
(60) Provisional application No. 61/659,333, filed on Jun. 13, 2012.

(51) Int. Cl. C12N 1/20; C02F 3/34; C12Q 1/68

(52) U.S. Cl. CPC C12N 1/20; C12Q 1/689; C02F 3/34
USPC 210/601; 435/252.1; 435/6.11; 435/6.12; 536/24.32; 536/24.33

(57) ABSTRACT

Provided are compositions comprising polyphosphate accumulating bacteria as well as a method for identifying said polyphosphate accumulating bacteria. Additionally provided is a method for treating various substances with said compositions.

### TECHNICAL FIELD

Provided are compositions comprising novel polyphosphate accumulating bacteria related to polyphosphate accumulating microbes. Further provided is a method for identifying said bacteria in a sample. Additionally provided is a method for treating various substances with said compositions.

### BACKGROUND

Excessive nutrient runoff, particularly phosphorus, in a lake or other body of water typically results in plant and algal blooms. Subsequent decomposition of these blooms depletes the supply of oxygen, creating anoxic conditions and death of animal life.

An important aspect of wastewater treatment is the removal of excess nutrients. The removal of phosphorus (P) from wastewater can be accomplished either by chemical precipitation or by a biological mechanism in a process named enhanced biological phosphorus removal (EBPR). The latter requires microorganisms known as polyphosphate-accumulating microorganisms or bacteria (PAOs), which can store phosphate as intracellular polyphosphate granules; therefore, removal of a portion of the growing biomass containing a high polyphosphate content (waste-activated sludge) results in the net removal of P from the wastewater (Oehmen et al., 2007). However, little is known of the genetics or biochemistry of the organisms responsible for polyphosphate-accumulation because they have not yet been isolated in pure culture, leading to poor stability and reliability of EBPR. Previous studies using 16S rRNA directed probes have identified the dominant polyphosphate-accumulating bacteria in acetate-fed laboratory scale sequencing batch reactors as members of the phylogenetically defined Rhodocyclus group in the β-proteobacteria (Crocetti et al., 2000, WO 01/46459). In addition, the Rhodocyclus related organisms have been repeatedly enriched and one such member is tentatively named Candidatus accumulibacter phosphatis (Hesselmann et al., 2000). The involvement of Accumulibacter-related organisms in EBPR was confirmed in full-scale EBPR wastewater treatment plants (Kong et al., 2004). Nevertheless, it is unlikely that Accumulibacter are the only phosphate accumulating bacteria groups in EBPR systems based on FISH studies that have observed poly-phosphate in other unrelated organisms in these communities (He et al., 2008). However, to date, it has not been possible to obtain pure cultures of polyphosphate accumulating bacteria.

Phosphorous has also been found to accumulate in solid waste as well. US Patent Publication No. 20120103037 discloses a method for treating solid waste with a combination of leaching and polyphosphate accumulating microorganisms.

There is clearly a need for an efficient system and/or effective composition for removing phosphates from various liquid and solid locations. It is an objective to provide such a system and/or compositions.

### SUMMARY OF DISCLOSURE

Provided is a composition comprising or population of one or more polyphosphate accumulating microorganisms, wherein said microorganism:

(a) comprises a nucleotide sequence, wherein said sequence has at least about 97% homology, identity or similarity to at least one of the nucleotide sequences set forth in SEQ ID NOs: 1-6;

SEQ ID NO: 1: >1492Plc8
ACGGGGGCAACCCTGGTGGCGAGTGGCGAACGGGTGAGTAATGCATCGGA
ACATACCCAGTCGTGGGGGATAACGTAGCGAAAGTTACGCTAATACCGCA
TACGTCCTGAGGGAGAAAGCGGGGGATCGCAAGACCTCGCGCGATTGGAG
TGGCCGATGTCAGATTAGCTAGTTGGTGGGGTAAAGGCCCACCAAGGCGA
CGATCTGTAGCGGGTCTGAGAGGATGATCCGCCACACTGGGACTGAGACA
CGGCCCAGACTCCTACGGGAGGCAGCAGTGGGGAATTTTGGACAATGGGG
GCAACCCTGATCCAGCCATGCCGCGTGCGGGAAGAAGGCCTTCGGGTTGT
AAACCGCTTTCGGACGGAAAGAAATCGCCATCTCTAACATAGGTGGTGGA
TGACGGTACCGTAAGAAGAAGCACCGGCTAACTACGTGCCAGCAGCCGCG
GTAATACGTAGGGTGCGAGCGTTAATCGGAATTACTGGGCGTAAAGCGTG
CGCAGGCGGTTTCTTAAGCCAGACGTGAAATCCCCGGGCTTAACCTGGGA
ACTGCGTTTGGAACTGGGAGACTAGAGTGTGTCAGAGGGAGGTGGAATTC
CGCGTGTAGCAGTGAAATGCGTAGATATGCGGAGGAACACCGATGGCGAA
GGCAGCCTCCTGGGATAACACTGACGCTCATGCACGAAAGCGTGGGGAGC
AAACAGGATTAGATACCCTGGTAGTCCACGCCCTAAACGATGTCAACTGG
CTGTTGGGAGAGAAATCTTTCAGTAGCGAAGCTAACGCGTGAAGTTGANC
GCCTGGGGAGTACGGCCGCAAGGTTGAAACTCAAAGGAATTGANGGGGCC
CGCACAAGCGGTGGATGATGTGGATTAATTCGATGCAACGCGAAGAACCT
TACCTACCCTTGACATGCCAGGAATCCCGNANAGATCTGGGGG
SEQ ID NO: 2
P4B5_8
TCGGNTCCNCTAGTAACGGCCGCCAGTGTGCTGGAATTCGCCCTTACGGG
GGCAACCCTGGTGGCGAGCGGCGAACGGGTGAGTAACACATCGGAACGTA
CCCTGTCGTGGGGGATAGCCCGGCGAAAGCCGGATTAATACCGCATACGA
CCTGAGGGTGAAAGCGGGGGATCGCAAGACCTCGCGCGATAGGAGCGGCC
GATGGCGGATTAGCTAGTTGGTGGGGTAAAGGCCTACCAAGGCGACGATC
CGTAGCTGGTCTGAGAGGACGACCAGCCACACTGGAACTGAGACACGGTC
CAGACTCCTACGGGAGGCAGCAGTGGGGAATTTTGGACAATGGGGGCAAC
CCTGGTCCAGCCATGCCGCGTGCGGGAAGAAGGCCTTCGGGTTGTAAACC
GCTTTCGGACAGAAAGAAATCGTTCGCTCTAACATAGCGGATGGATGACG
GTACTGTAAGAAGAAGCACCGGCTAACTACGTGCCAGCAGCCGCGGTAAT
ACGCAGGGTGCGAGCGTTAATCGGAATTACTGGGCGTAAAGCGTGCGCAG
GCGGATATGTAAGTCAGACGTGAAATCCCCGGGCTTAACCTGGGAATTGC
GTTTGAAACTGTATATCTAGAGTGCGTCAGAGGGGGGTGGAATTCCACGT
GTAGCAGTGAAATGCGTAGATATGCGGAGGAACACCGATGGCGAAGGCAA
TCCCCTGGGCCTGTACTGACGCTCATGCACGAAAGCGTGGGGAGCAAACA
GGATTAGATACCCTGGTAGTCCACGCCCTAAACGATGTCAACTGGCTGTT
GGGTGCGAGAGTACTCAGTAGCGAAGCTAACGCGTGAAGTTGACCGCCTG
GGGAGTACGGCCGCAAGGTTGAAACTCAAAGGAATTGACGGGGACCCGCA
CAAGCGGTGGATGATGTGGTTTAATTCGATGCAACGCGAAAAACCTTACC
CACCTTTGACATGTACGGAAT
SEQ ID NO: 3
P4B5_1
CTAGTAACGGCCGCCAGTGTGCTGGAATTCGCCCTTACGGGGGCAACCCT
GGTCCAGCCATGCCGCGCGCGGGAAGAAGGCCTTCGGGTTGTAAACCGCT
TTCGGACAGAAAGAAATCGTTCGCTCTAACATAGCGGATGGATGACGGTA
CTGTAAGAAGAAGCACCGGCTAACTACGTGCCAGCAGCCGCGGTAATACG
TAGGGTGCGAGCGTTAATCGGAATTACTGGGCGTAAAGCGTGCGCAGGCG
GATATGTAAGTCAGACGTGAAATCCCCGGGCTTAACCTGGGAATTGCGTT
TGAAACTGTATATCTAGAGTGCGTCAGAGGGGGGTGGAATTCCACGTGTA
GCAGTGAAATGCGTAGAGATGTGGAGGAACACCGATGGCGAAGGCAGCCC
CCTGGGATGACACTGACGCTCATGCACGAAAGCGTGGGGAGCAAACAGGA
TTAGATACCCGGGTAGTCCACGCCCTAAACGATGTCAACTGGCTGTTGGG
TGCGAGAGTACTCAGTAGCGAAGCTAACGCGTGAAGTTGACCGCCTGGGG
AGTACGGCCGCAAGGTTGAAACTCAAAGGAATTGACGGGGGCCCGCACAA
GCGGTGGATGATGTGGATTAATTCGATGCAACGCGAAGAACCTTACCTAC
CCTTGACATGCCAGGAACTTGCCAGAGATGGCTTGGTGCCCGAAAGGGAA
CCTGGACACAGGTGCTGCAAGGCTGTCGTCAGCTCGTGTCGTGAGATGTT
GGGTTAAGTCCCGCAACGAGCGCAACCCTTGTCATTAGTTGC
SEQ ID NO: 4
>1492Plc12
ACGGGGGCAACCCTGGTGGCGAGTGGCGAACGGGTGAGTAATGCATCGGA
ACATACCCAGTCGTGGGGGATAACGTAGCGAAAGTTACGCTAATACCGCA
TACGTCCTGAGGGAGAAAGCGGGGGATCGCAAGACCTCGCGCGATTGGAG
TGGCCGATGTCAGATTAGCTAGCTGGTGGGGTAAAGGCCCACCAAGGCGA
CGATCTGTAGCGGGTCTGAGAGGATGATCCGCCACACTGGGACTGAGACA
CGGCCCAGACTCCTACGGGAGGCAGCAGTGGGGAATTTTGGACAATGGGG
GCAACCCTGATCCAGCCATGCCGCGTGCGGGAAGAAGGCCTTCGGGTTGT
AAACCGCTTTCGGACGGAAAGAAATCGCCATCTCTAACATAGGTGGTGGA
TGACGGTACCGTAAGAAGAAGCACCGGCTAACTACGTGCCAGCAGCCGCG
GTAATACGTAGGGTGCGAGCGTTAATCGGAATTACTGGGCGTAAAGCGTG
CGCAGGCGGTTTCTTAAGCCAGACGTGAAATCCCCGGGCTTAACCTGGGA
ACTGCGTTTGGAACTGGGAGACTAGAGTGTGTCAGAGGGAGGTGGAATTC
CGCGTGTAGCAGTGAAATGCGTAGATATGCGGAGGAACACCGATGGCGAA
GGCAGCCTCCTGGGATAACACTGACGCTCATGCACGAAAGCGTGGGGAGC
AAACAGGATTAGATACCCTGGTAGTCCACGCCCTAAACGATGTCAACTGG
CTGTTGGGAGAGAAATCTTTCAGTAGCGAAGCTAACGCGTGAAGTTGACC
GCCTGGGGAGTACGGCCGCAAGGTTGAAACTCAAAGGAATTGANGGGGCC
CGCACAAGCGGTGGATGATGTGGATTAATTCGATGCAACGCGAAGAACCT
TACCTACCCTTGACATGCCAGGAATCCCGGAGAGATCTGGGGG
SEQ ID NO: 5
>1492Plc1
CCTGGTGGCGAGTGGCGAACGGGTGAGTAATGCATCGGAACATACCCAGT
CGTGGGGGATAACGTAGCGAAAGTTACGCTAATACCGCATACGTCCTGAG
GGAGAAAGCGGGGGATCGCAAGACCTCGCGCGATTGGAGTGGCCGATGTC
AGATTAGCTAGTTGGTGGGGTAAAGGCCCACCAAGGCGACGATCTGTAGC
GGGTCTGAGAGGATGATCCGCCACACTGGGACTGAGACACGGCCCAGACT
CCTACGGGAGGCAGCAGTGGGGAATTTTGGACAATGGGGGCAACCCTGAT
CCAGCCATGCCGCGTGCGGGAAGAAGGCCTTCGGGTTGTAAACCGCTTTC
GGACGGAAAGAAATCGCCATCTCTAACATAGGTGGTGGATGACGGTACCG
TAAGAAGAAGCACCGGCTAACTACGTGCCAGCAGCCGCGGTAATACGTAG
GGTGCGAGCGTTAATCGGAATTACTGGGCGTAAAGCGTGCGCAGGCGGTT
TCTTAAGCCAGACGTGAAATCCCCGGGCTTAACCTGGGAACTGCGTTTGG
AACTGGGAGACTAGAGTGTGTCAGAGGGAGGTGGAATTCCGCGTGTAGCA
GTGAAATGCGTAGATATGCGGAGGAACACCGATGGCGAAGGCAGCCTCCT
GGGATAACACTGACGCTCATGCACGAAAGCGTGGGGAGCAAACAGGATTA
GATACCCTGGTAGTCCACGCCCTAAACGATGTCAACTGGCTGTTGGGAGA
GAAATCTTTCAGTAGCGAAGCTAACGCGTGAAGTTGACCGCCTGGGGAGT
ACGGCCGCAAGGTTGAAACTCANGGAATTGACGGGGGCCCGCACAAGCGG
TGGATGATGTGGATTAATTCGATGCAACGCGAAGAACCTTACCTACCCTN
GACATGCCAGGAATCCCGGAGANATCTGGGGG
SEQ ID NO: 6
>1492Plc6
ACGGGGGCAACCCTGGTGGCGAGTGGCGAACGGGTGAGTAATGCATCGGA
ACATACCCAGTCGTGGGGGATAACGTAGCGAAAGTTACGCTAATACCGCA
TACGTCCTGAGGGAGAAAGCGGGGGATCGCAAGACCTCGCGCGATTGGAG
TGGCCGATGTCAGATTAGCTAGTTGGTGGGGTAAAGGCCCACCAAGGCGA
CGATCTGTAGCGGGTCTGAGAGGATGATCCGCCACACTGGGACTGAGACA
CGGCCCAGACTCCTACGGGAGGCAGCAGTGGGGAATTTTGGACAATGGGG
GCAACCCTGATCCAGCCATGCCGCGTGCGGGAAGAAGGCCTTCGGGTTGT
AAACCGCTTTCGGACGGAAAGAAATCGCCATCTCTAACATAGGTGGTGGA
TGACGGTACCGTAAGAAGAAGCACCGGCTAACTACGTGCCAGCAGCCGCG
GTAATACGTAGGGTGCGAGCGTTAATCGGAATTACTGGGCGTAAAGCGTG
CGCAGGCGGTTTCTTAAGCCAGACGTGAAATCCCCGGGCTTAACCTGGGA
ACTGCGTTTGGAACTGGGAGACTAGAGTGTGTCAGAGGGAGGTGGAATTC
CGCGTGTAGCAGTGAAATGCGTAGATATGCGGAGGAACACCGATGGCGAA
NGCAGCCTCCTGGGATAACACTGACGCTCATGCACGAAAGCGTNNGAGCA
AACAGGATTAGATACCCTGGTAGTCCACGCCCTAAACGATGTCAACTGGC
TGTTGGGAGAGAAATCTTTCAGTAGCGAAGCTAACGCGTGAAGTTGACCG
CCTGGGGAGTACGGCCGCAAGGTTGAAACTCAAAGGAATTGACGGGGGCC
CGCACAAGCGGTGGATGATGTGGATTAATTCGATGCAACGCGAAGAACCT
TACCTACCCTTGACATGCCAGGAATCCCGGAGANATCTGGGGG

and

(b) comprises nucleotide sequences having about 90-95% homology, identity or similarity to Rhodocyclus tenuis DSM110 and/or Candidatus accumulibacter.

In a particular embodiment, the composition is obtainable from a bioreactor. In another particular embodiment, the polyphosphate accumulating microorganisms are bacteria (PAB). The composition of PAB may be used to reduce the amount of inorganic phosphate in a waste-stream including but not limited to sewage treatment plant effluent, solid waste, sludge, agricultural drainage and industrial effluent.

In a related aspect, also provided is a method of identifying these polyphosphate accumulating microorganisms which comprises contacting a sample with one or more probe or primers comprising a sequence, wherein said sequence has at least about 97% homology, identity or similarity to a nucleotide sequence selected from the group consisting of: SEQ ID NOS: 9-10:

| | | |
|---|---|---|
| Rcyc_69F: | ACGGGGGCAACCCTGGT; | (SEQ ID NO: 9) |
| Rcyc_149F: | ATAACCTGGCGAAAGCCAGG | (SEQ ID NO: 10) |

and detecting the presence or absence of said polyphosphate accumulating microorganisms.

The method may also further comprise contacting the sample with one or more probes or primers comprising sequences depicted in SEQ ID NOS: 7, 8, 11 or 12:

| | |
|---|---|
| 27F: | |
| AGAGTTTGATCCTGGCTCAG; | (SEQ ID NO: 7) |
| 1492R: | |
| GGTTACCTTGTTACGACTT; | (SEQ ID NO: 8) |
| Rcyc_168R: | |
| CCTGGCTTTCGCCAGGTTAT | (SEQ ID NO: 11) |
| and | |
| Rcyc_1446R: | |
| CTACCAGAAGCAGTTAGCCTA. | (SEQ ID NO: 12) |

Further provided are the oligonucleotide probes or primers at least 17 nucleotides in length comprising SEQ ID NOS: 9-10 or their complementary sequences and which bind to polyphosphate accumulating microorganism 16S rRNA sequences as well as kits comprising one or more of these sequences. The kits may further comprise SEQ ID NOs: 7, 8, 11 and/or 12.

Additionally provided is a method for obtaining these compositions. The steps comprise:

(a) providing a bioreactor capable of containing, for example, one or more polyphosphate accumulating bacteria (PAB) which (i) comprises a nucleotide sequence, wherein said sequence has at least about 97% homology, identity or similarity to at least one of the nucleotide sequences set forth in SEQ ID NOs: 1-6 and (ii) comprises nucleotide sequences having about 90-95% homology, identity or similarity to Rhodocyclus tenuis DSM110 and/or Candidatus accumulibacter;

(b) identifying polyphosphate accumulating bacteria in said bioreactor of (a) and

(c) obtaining a composition containing an amount of polyphosphate accumulating bacteria identified in (b) sufficient to decrease the presence of inorganic phosphate from a location.

As will be set forth in further detail below, polyphosphate accumulating bacteria may be identified by determining if there bacteria containing any of the nucleotide sequences having at least about 97% identity to a sequence, wherein said sequence is at least one of SEQ ID NOs: 1-6. Once such bacteria are identified, they may further be tested for various metabolic properties such as the ability to accumulate P. The bacteria obtained may be formulated into compositions that may be used to develop a treatment or production process that applies these polyphosphate accumulating bacteria.

Provided is a method for decreasing the amount of dissolved inorganic phosphate in an effluent in need thereof comprising applying the composition set forth above in an amount effective to reduce said inorganic phosphate in said effluent. The phosphate may be reduced by greater than about 80%. The effluent may be P-laden waste which includes but is not limited to waste water, sewage sludge or agricultural drainage.

### BRIEF DESCRIPTION OF THE FIGURES

FIG. 1 shows a phylogenetic tree of 16S rDNA sequences obtained from bioreactor samples, P4B5 and Plc, in this study and reference sequences from NCBI database. Actinomyces sp. BL-79 was used as the out group. Bootstrap scores (%) were also shown.

### DETAILED DESCRIPTION

While the compositions and methods heretofore are susceptible to various modifications and alternative forms, exemplary embodiments will herein be described in detail. It should be understood, however, that there is no intent to limit the invention to the particular forms disclosed, but on the contrary, the intention is to cover all modifications, equivalents, and alternatives falling within the spirit and scope of the invention as defined by the appended claims.

Where a range of values is provided, it is understood that each intervening value, to the tenth of the unit of the lower limit unless the context clearly dictates otherwise, between the upper and lower limit of that range and any other stated or intervening value in that stated range, is included therein. Smaller ranges are also included. The upper and lower limits of these smaller ranges are also included therein, subject to any specifically excluded limit in the stated range.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

As defined herein, “derived from” means directly isolated or obtained from a particular source or alternatively having identifying characteristics of a substance or organism isolated or obtained from a particular source.

The terms “polynucleotide(s)”, “nucleic acid molecule(s)” and “nucleic acids” will be used interchangeably.

The terms “percent homology”, “percent similarity” and “percent identity” are used interchangeably.

“Percent Identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison, and multiplying the result by 100 to yield the percentage of sequence identity.

Optimal alignment of sequences for comparison can use any means to analyze sequence identity (homology) known in the art, e.g., by the progressive alignment method of termed “PILEUP” (Morrison, 1997), as an example of the use of PILEUP); by the local homology algorithm of Smith & Waterman, (1981); by the homology alignment algorithm of Needleman & Wunsch (1970); by the search for similarity method of Pearson (1988); by computerized implementations of these algorithms (e.g., GAP, BEST FIT, FASTA, and TFASTA in the Wisconsin Genetics Software Package, Genetics Computer Group, 575 Science Dr., Madison, Wis.); ClustalW (CLUSTAL in the PC/Gene program by Intelligenetics, Mountain View, Calif., described by, e.g., Higgins (1988); Corpet (1988); Huang (1992); and Pearson (1994); Pfamand Sonnhammer (1998); TreeAlign (Hein (1994); MEG-ALIGN, and SAM sequence alignment computer programs; or, by manual visual inspection.

Another example of an algorithm that is suitable for determining sequence similarity is the BLAST algorithm, which is described in Altschul et al., (1990). The BLAST programs (Basic Local Alignment Search Tool) of Altschul, S. F., et al., (1993) searches under default parameters for identity to sequences contained in the BLAST “GENEMBL” database. A sequence can be analyzed for identity to all publicly available DNA sequences contained in the GENEMBL database using the BLASTN algorithm under the default parameters. Software for performing BLAST analyses is publicly available through the National Center for Biotechnology Information,www.ncbi.nlm.nih.gov/; see also Zhang (1997) for the “PowerBLAST” variation. This algorithm involves first identifying high scoring sequence pairs (HSPs) by identifying short words of length W in the query sequence that either match or satisfy some positive valued threshold score T when aligned with a word of the same length in a database sequence. T is referred to as the neighborhood word score threshold (Altschul et al., (1990)). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a wordlength (W) of 11, the BLOSUM62 scoring matrix (see Henikoff (1992)) alignments (B) of 50, expectation (E) of 10, M=5, N=−4, and a comparison of both strands. The term BLAST refers to the BLAST algorithm which performs a statistical analysis of the similarity between two sequences; see, e.g., Karlin (1993).

### Identification of Polyphosphate Accumulating Bacteria

The bacterium may be identified in a sample using methods set forth below. In a particular embodiment, the sample may be obtained from e.g. a bioreactor using methods set forth in application serial no. PC T/US 2012/060010. In a particular embodiment, the sample may be derived from products from the methods set forth in application serial nos. PCT/US 2012/060010. In yet another particular embodiment, the sample may be derived from SoilBuilder™ sold by Agricen, Pilot Point, Tex. SoilBuilder™ contains bacteria and bacterial metabolites derived from the bioreactor. Based on plate counts using tryptic soy agar (TSA) (incubation for 24 h at 25 C). the most commonly occurring bacteria within the I111al stabilized product are Acidovoras bacillus, Bacillus licheniformis, Bacillus subtilis, Bacillus oleronius, Bacillus marinus, Bacillus megaterium, and Rhodococcus rhodochrous, each at 1×10³ colony-forming units (cfu) mL⁻¹.

In yet even another particular embodiment, the sample may be further enriched for polyphosphate accumulated microorganisms, by for example, aerobic/anaerobic cycling (see, for example, Coats et al., 2011) or the aerobic/extended idle protocol (see, for example, Wang, 2012) or alternatively the sample may be further cultivated using an in situ cultivation protocol (see, for example, Bollman et al., 2007).

A nucleic acid (e.g., DNA) may be obtained from a sample from, using methods known in the art (e.g., nucleic acid extraction). This nucleic acid may be hybridized to a probe or primer using methods known in the art.

Alternatively, the probe or primer may act as primer for amplification in, for example, a PCR reaction. In a particular embodiment, the probe may comprise a nucleotide sequence having at least about 97% identity to SEQ ID NOS:7, 8, 9 or 10. In more particular embodiments, the probe or primer comprises a nucleotide sequence that has greater than about 97%, 98%, 99%, or 99.5% identity to SEQ ID NOS: 7, 8, 9 or 10. In more particular embodiment, the probe or primer comprises a nucleotide sequence that has greater than 97% identity to SEQ ID NOS: 9 or 10. In yet another specific embodiment, the probe may comprise SEQ ID NO: 9 or 10. Alternatively, a primer(s) universal to substantially all 16S rRNA sequences may be used in addition to the primers set forth above and may include but is not limited to SEQ ID NOS: 7, 8, 11 and 12. The probes or primers are at least 17 nucleotides in length and may range from about 17 nucleotides in length to about 200 nucleotides.

The PCR reaction products in the sample may be compared with 16S rRNA or rDNA sequences of related polyphosphate accumulating bacteria using various methods known in the art, including but not limited to BLAST, the Ribosomal Database Project, or Fluorescent in situ Hybridization (FISH) analysis using methods known in the art. In a preferred embodiment, the samples should comprise polynucleotide sequences having between about 90-95% identity to Rhodocyclus tenuis DSM110 and Candidatus accumulibacter 16S rDNA or rRNA sequences as well as sequences having at least about 97% identity to at least one of SEQ ID NOs: 1-6. In more particular embodiments, the probe or primer comprises a nucleotide sequence that has greater than about 97%, 98%, 99%, or 99.5% identity to SEQ ID NOS: 1-6. The amount of polyphosphate accumulating microorganisms could be determined as well.

The samples may be further tested for the presence of polyphosphate accumulating bacteria using methods known in the art by, for example, testing the samples for the ability to remove phosphate from various liquid or solid waste samples. Optionally, samples may be enriched for polyphosphate accumulating bacteria before and/or after further testing.

The probes or primers used may be packaged into test kits. These kits may further contain detectable labels and written instructions. In a particular embodiment, the probes or primers may be attached to solid supports.

### Compositions and Uses

Samples containing the requisite polyphosphate accumulating bacteria are formulated into compositions. In a particular embodiment, the samples may be cultured under conditions to enrich for the requisite polyphosphate accumulating bacteria. In a specific embodiment, the polyphosphate accumulating bacteria comprise at least one of the polynucleotide sequences set forth in SEQ ID NOS: 1-6 and may be present in the amount of greater than about 25% by weight.

The compositions may be used to reduce or remove inorganic phosphate from various locations including but not limited, to effluents such as wastewater and/or solid waste such as sewage sludge and agricultural drainage. In a specific embodiment, the compositions may be applied to wastewater in amounts effective to decrease the amount of phosphorous present by at least about 80%.

### Example

The composition and methods set forth above will be further illustrated in the following, non-limiting Examples. The examples are illustrative of various embodiments only and do not limit the claimed invention regarding the materials, conditions, weight ratios, process parameters and the like recited herein.

### Methods

### DNA Extraction

DNA was extracted from 10 ml of bioreactor samples with the FastDNA Spin Kit (MP Bio), according to the protocol of the manufacturer. The quantity of the DNA extractions was checked by Nanodrop.

### Clone Libraries

Bacterial 16S rDNA clone libraries were constructed from extracted genomic DNA. Briefly, combinations of universal (27F (SEQ ID NO: 7) and 1492R (SEQ ID NO:8)) and Rhodocyclus specific (Rcyc 69F, 168R, 149F, and 1446R) primers were used for PCR amplification and, amplified 16S rDNA genes were cloned using a TOPO TA cloning kit (Invitrogen).

16S rRNA Gene Retrieval and Phylogenetic Analysis

Thirty clones from the libraries were picked randomly, and TOPO plasmids harboring 16S rDNA gene were isolated using 5 PRIME Fast Plasmid Mini kit. The retrieved sequences were classified using RDP database and compared with a reference 16S rRNA using Blast. The phylogenetic tree was created by MEGA 5.1 using neighbor joining and bootstrap analysis. Primers prepared are shown below:

| | | |
|---|---|---|
| 27F: | AGAGTTTGATCCTGGCTCAG | (SEQ ID NO: 7) |
| 1492R: | GGTTACCTTGTTACGACTT | (SEQ ID NO: 8) |
| Rcyc_69F: | ACGGGGGCAACCCTGGT | (SEQ ID NO: 9) |
| Rcyc_149F: | ATAACCTGGCGAAAGCCAGG | (SEQ ID NO: 10) |
| Rcyc_168R: | CCTGGCTTTCGCCAGGTTAT | (SEQ ID NO: 11) |
| Rcyc_1446R: | CTACCAGAAGCAGTTAGCCTA | (SEQ ID NO: 12) |

### Results

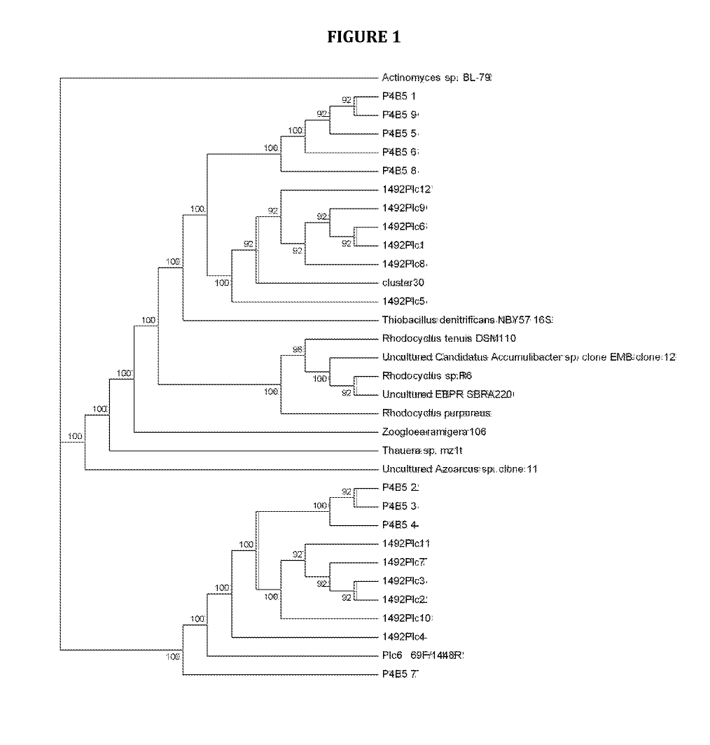

21 clones from the library generated by Rcyc_{—}69F (SEQ ID NO:9) and 1492R (SEQ ID NO:8) primers were grouped into unclassified Rhodocyclaceae family. From phylogenetic tree analysis, 7 clones of the family were found to be closely related to previous polyphosphate-accumulating Rhodocyclus group members (FIG. 1). In fact, they showed similarity ranging from 90-94% to Rhodocyclus tenuis and Candidatus Accumulibacter sp. (Table 1).

TABLE 1
Similarities of closely related Rhodocyclus clones to
Rhodocyclus tenuis DSM110 and Candidatus Accumulibacter sp.

similarity to (%)

| | Rhodocyclus tenuis | Candidatus |
|---|---|---|
| Clones | DSM110 | Accumulibacter sp. |
| 1942Plc8 | 94 | 93 |
| (SEQ ID NO: 1) | | |
| P4B5 8 | 92 | 90 |
| (SEQ ID NO: 2) | | |
| P4B5 1 | 91 | 90 |
| (SEQ ID NO: 3) | | |
| 1492Plc 12 | 91 | 90 |
| (SEQ ID NO: 4) | | |
| 1492Plc 1 | 91 | 90 |
| (SEQ ID NO: 5) | | |
| 1492Plc 6 | 91 | 90 |
| (SEQ ID NO: 6) | | |

This invention may be embodied in other forms or carried out in other ways without departing from the spirit or essential characteristics thereof. The present disclosure is therefore to be considered as in all aspects illustrate and not restrictive, and all changes which come within the meaning and range of equivalency are intended to be embraced therein.

Various references are cited throughout this specification, each of which is incorporated herein by reference in its entirety.

### REFERENCE LIST

Altschul et al. (1993) J. Mol. Biol. 219: 555-565.
Altschul et al. (1990) J. Mol. Biol. 215: 403-410.
Bollman et al. (2007) Appl. Environ. Microbiol. 73:6386-6390.
Coats et al. (2011) Water Environ. Res. 83:461-470.
Corpet (1988) Nucleic Acids Res. 16:10881-10890.
Crocetti et al. (2000) Appl. Environ. Microbiol. 66:1175-1182.
He et al. (2008) Microb. Ecol. 55:229-236.
Hesselmann et al. (2000) Syst. Appl. Microbiol. 22:454-465.
Hein (1994) Methods Mol. Biol. 25:349-364.
Higgins (1988) Gene 73: 237-244.
Huang (1992) Comp. Appl. Biosci. 8:155-165.
Karlin (1993) Proc. Natl. Acad. Sci. USA 90:5873-5787.
Kong et al. (2004) Appl. Environ. Microbiol. 70:5383-5390.
Morrison (1997) Mol. Biol. Evol. 14:428-441.
Needleman and Wunsch (1970) J. Mol. Biol. 48:443-453.
Oehmen et al. (2008) Water Research 41:2271-2300.
Pearson and Lipmann (1988) Proc. Natl. Acad. Sci. USA 85: 2444-2448.
Pearson (1994) Methods in Mol. Biol. 24:307-331.
Pfam et al. (1998) Nucleic Acids Res. 26:322-325.
Smith and Waterman (1981) J. Mol. Biol. 147:195-197.
Wang (2012) Water Res. 46:3868-3878.
Zhang (1997) Genome Res. 7:649-656.
Zilles et al. (2002) Appl. Environ. Microbiol. 68:2763-2769.

What is claimed is:

1. A composition comprising or population of one or more polyphosphate accumulating bacteria, wherein said bacteria:
(a) comprises one or more nucleotide sequences, wherein said sequence has at least about 97% identity to at least one of the nucleotide sequences set forth in SEQ ID NOs: 1-6 and
(b) comprises nucleotide sequences having about 90-95% homology to Rhodocyclus tenuis DSM110 and/or Candidatus accumulibacter.

2. The composition according to claim 1, wherein said composition is obtainable from a sample.

3. The composition according to claim 2, wherein said sample is obtainable or derived from a bioreactor.

4. A method for identifying one or more polyphosphate accumulating bacteria in a sample comprising contacting said sample with one or more probe or primers having at least about 97% homology to a nucleotide sequence, wherein said nucleotide sequence is selected from the group consisting of: SEQ ID NOS: 9-10 and detecting the presence of absence of said polyphosphate accumulating bacteria.

5. The method according to claim 4, wherein said method further comprises contacting the sample with one or more probes or primers selected from the group consisting of SEQ ID NOS: 7, 8, 11, 12.

6. An oligonucleotide probe or primer for detecting a polyphosphate accumulating bacteria having a sequence of at least 17 nucleotides with at least about 97% homology to a nucleotide sequence, wherein said sequence is selected from the group consisting of SEQ ID NOS: 9-10.

7. A kit comprising one or more oligonucleotides of claim 6.

8. The kit of claim 7, which further comprises one or more probes or primers selected from the group consisting of SEQ ID NOS: 7, 8, 9-10.

9. A method for obtaining the composition or population of claim 1, comprising
(a) providing a sample capable of containing one or more polyphosphate accumulating bacteria which (i) comprises a nucleotide sequence, wherein said sequence has at least about 97% homology, identity or similarity to at least one of the nucleotide sequences set forth in SEQ ID NOs: 1-6 and (ii) comprises nucleotide sequences having about 90-95% homology, identity or similarity to Rhodocyclus tenuis DSM110 and Candidatus accumulibacter;
(b) identifying one or more polyphosphate accumulating bacteria in said substance; and
c) obtaining a composition containing an amount of polyphosphate accumulating bacteria sufficient to decrease the presence of inorganic phosphate from a location where there is a need for such a reduction.

10. The method according to claim 9, wherein said sample is derived or obtainable from a bioreactor.

11. The method according to claim 10, wherein said sample derived or obtainable from said bioreactor is SoilBuilder™ (Agricen, Pilot Point, Tex.).

12. A method for decreasing the amount of inorganic phosphate in an effluent in need thereof comprising applying the composition of claim 1 in an amount effective to reduce said inorganic phosphate in said effluent.

13. The method according to claim 11, wherein said effluent is derived from waste-water, sewage sludge, industrial waste, agricultural waste.


SEQUENCE LISTING (12 sequences)

SEQ ID NO: 1 - DNA, 943 bp, Unknown
  1492Plc8
acgggggcaaccctggtggcgagtggcgaacgggtgagtaatgcatcggaacatacccagtcgtgggggataacgtagcgaaagttacgctaataccgcatacgtcctgagggagaaagcgggggatcgcaagacctcgcgcgattggagtggccgatgtcagattagctagttggtggggtaaaggcccaccaaggcgacgatctgtagcgggtctgagaggatgatccgccacactgggactgagacacggcccagactcctacgggaggcagcagtggggaattttggacaatgggggcaaccctgatccagccatgccgcgtgcgggaagaaggccttcgggttgtaaaccgctttcggacggaaagaaatcgccatctctaacataggtggtggatgacggtaccgtaagaagaagcaccggctaactacgtgccagcagccgcggtaatacgtagggtgcgagcgttaatcggaattactgggcgtaaagcgtgcgcaggcggtttcttaagccagacgtgaaatccccgggcttaacctgggaactgcgtttggaactgggagactagagtgtgtcagagggaggtggaattccgcgtgtagcagtgaaatgcgtagatatgcggaggaacaccgatggcgaaggcagcctcctgggataacactgacgctcatgcacgaaagcgtggggagcaaacaggattagataccctggtagtccacgccctaaacgatgtcaactggctgttgggagagaaatctttcagtagcgaagctaacgcgtgaagttgancgcctggggagtacggccgcaaggttgaaactcaaaggaattganggggcccgcacaagcggtggatgatgtggattaattcgatgcaacgcgaagaaccttacctacccttgacatgccaggaatcccgnanagatctggggg

SEQ ID NO: 2 - DNA, 971 bp, Unknown
  P4B5_8
tcggntccnctagtaacggccgccagtgtgctggaattcgcccttacgggggcaaccctggtggcgagcggcgaacgggtgagtaacacatcggaacgtaccctgtcgtgggggatagcccggcgaaagccggattaataccgcatacgacctgagggtgaaagcgggggatcgcaagacctcgcgcgataggagcggccgatggcggattagctagttggtggggtaaaggcctaccaaggcgacgatccgtagctggtctgagaggacgaccagccacactggaactgagacacggtccagactcctacgggaggcagcagtggggaattttggacaatgggggcaaccctggtccagccatgccgcgtgcgggaagaaggccttcgggttgtaaaccgctttcggacagaaagaaatcgttcgctctaacatagcggatggatgacggtactgtaagaagaagcaccggctaactacgtgccagcagccgcggtaatacgcagggtgcgagcgttaatcggaattactgggcgtaaagcgtgcgcaggcggatatgtaagtcagacgtgaaatccccgggcttaacctgggaattgcgtttgaaactgtatatctagagtgcgtcagaggggggtggaattccacgtgtagcagtgaaatgcgtagatatgcggaggaacaccgatggcgaaggcaatcccctgggcctgtactgacgctcatgcacgaaagcgtggggagcaaacaggattagataccctggtagtccacgccctaaacgatgtcaactggctgttgggtgcgagagtactcagtagcgaagctaacgcgtgaagttgaccgcctggggagtacggccgcaaggttgaaactcaaaggaattgacggggacccgcacaagcggtggatgatgtggtttaattcgatgcaacgcgaaaaaccttacccacctttgacatgtacggaat

SEQ ID NO: 3 - DNA, 792 bp, Unknown
  P4B5_1
ctagtaacggccgccagtgtgctggaattcgcccttacgggggcaaccctggtccagccatgccgcgcgcgggaagaaggccttcgggttgtaaaccgctttcggacagaaagaaatcgttcgctctaacatagcggatggatgacggtactgtaagaagaagcaccggctaactacgtgccagcagccgcggtaatacgtagggtgcgagcgttaatcggaattactgggcgtaaagcgtgcgcaggcggatatgtaagtcagacgtgaaatccccgggcttaacctgggaattgcgtttgaaactgtatatctagagtgcgtcagaggggggtggaattccacgtgtagcagtgaaatgcgtagagatgtggaggaacaccgatggcgaaggcagccccctgggatgacactgacgctcatgcacgaaagcgtggggagcaaacaggattagatacccgggtagtccacgccctaaacgatgtcaactggctgttgggtgcgagagtactcagtagcgaagctaacgcgtgaagttgaccgcctggggagtacggccgcaaggttgaaactcaaaggaattgacgggggcccgcacaagcggtggatgatgtggattaattcgatgcaacgcgaagaaccttacctacccttgacatgccaggaacttgccagagatggcttggtgcccgaaagggaacctggacacaggtgctgcaaggctgtcgtcagctcgtgtcgtgagatgttgggttaagtcccgcaacgagcgcaacccttgtcattagttgc

SEQ ID NO: 4 - DNA, 943 bp, Unknown
  1492Plc12
acgggggcaaccctggtggcgagtggcgaacgggtgagtaatgcatcggaacatacccagtcgtgggggataacgtagcgaaagttacgctaataccgcatacgtcctgagggagaaagcgggggatcgcaagacctcgcgcgattggagtggccgatgtcagattagctagctggtggggtaaaggcccaccaaggcgacgatctgtagcgggtctgagaggatgatccgccacactgggactgagacacggcccagactcctacgggaggcagcagtggggaattttggacaatgggggcaaccctgatccagccatgccgcgtgcgggaagaaggccttcgggttgtaaaccgctttcggacggaaagaaatcgccatctctaacataggtggtggatgacggtaccgtaagaagaagcaccggctaactacgtgccagcagccgcggtaatacgtagggtgcgagcgttaatcggaattactgggcgtaaagcgtgcgcaggcggtttcttaagccagacgtgaaatccccgggcttaacctgggaactgcgtttggaactgggagactagagtgtgtcagagggaggtggaattccgcgtgtagcagtgaaatgcgtagatatgcggaggaacaccgatggcgaaggcagcctcctgggataacactgacgctcatgcacgaaagcgtggggagcaaacaggattagataccctggtagtccacgccctaaacgatgtcaactggctgttgggagagaaatctttcagtagcgaagctaacgcgtgaagttgaccgcctggggagtacggccgcaaggttgaaactcaaaggaattganggggcccgcacaagcggtggatgatgtggattaattcgatgcaacgcgaagaaccttacctacccttgacatgccaggaatcccggagagatctggggg

SEQ ID NO: 5 - DNA, 932 bp, Unknown
  1492Plc1
cctggtggcgagtggcgaacgggtgagtaatgcatcggaacatacccagtcgtgggggataacgtagcgaaagttacgctaataccgcatacgtcctgagggagaaagcgggggatcgcaagacctcgcgcgattggagtggccgatgtcagattagctagttggtggggtaaaggcccaccaaggcgacgatctgtagcgggtctgagaggatgatccgccacactgggactgagacacggcccagactcctacgggaggcagcagtggggaattttggacaatgggggcaaccctgatccagccatgccgcgtgcgggaagaaggccttcgggttgtaaaccgctttcggacggaaagaaatcgccatctctaacataggtggtggatgacggtaccgtaagaagaagcaccggctaactacgtgccagcagccgcggtaatacgtagggtgcgagcgttaatcggaattactgggcgtaaagcgtgcgcaggcggtttcttaagccagacgtgaaatccccgggcttaacctgggaactgcgtttggaactgggagactagagtgtgtcagagggaggtggaattccgcgtgtagcagtgaaatgcgtagatatgcggaggaacaccgatggcgaaggcagcctcctgggataacactgacgctcatgcacgaaagcgtggggagcaaacaggattagataccctggtagtccacgccctaaacgatgtcaactggctgttgggagagaaatctttcagtagcgaagctaacgcgtgaagttgaccgcctggggagtacggccgcaaggttgaaactcanggaattgacgggggcccgcacaagcggtggatgatgtggattaattcgatgcaacgcgaagaaccttacctaccctngacatgccaggaatcccggaganatctggggg

SEQ ID NO: 6 - DNA, 943 bp, Unknown
  1492Plc6
acgggggcaaccctggtggcgagtggcgaacgggtgagtaatgcatcggaacatacccagtcgtgggggataacgtagcgaaagttacgctaataccgcatacgtcctgagggagaaagcgggggatcgcaagacctcgcgcgattggagtggccgatgtcagattagctagttggtggggtaaaggcccaccaaggcgacgatctgtagcgggtctgagaggatgatccgccacactgggactgagacacggcccagactcctacgggaggcagcagtggggaattttggacaatgggggcaaccctgatccagccatgccgcgtgcgggaagaaggccttcgggttgtaaaccgctttcggacggaaagaaatcgccatctctaacataggtggtggatgacggtaccgtaagaagaagcaccggctaactacgtgccagcagccgcggtaatacgtagggtgcgagcgttaatcggaattactgggcgtaaagcgtgcgcaggcggtttcttaagccagacgtgaaatccccgggcttaacctgggaactgcgtttggaactgggagactagagtgtgtcagagggaggtggaattccgcgtgtagcagtgaaatgcgtagatatgcggaggaacaccgatggcgaangcagcctcctgggataacactgacgctcatgcacgaaagcgtnngagcaaacaggattagataccctggtagtccacgccctaaacgatgtcaactggctgttgggagagaaatctttcagtagcgaagctaacgcgtgaagttgaccgcctggggagtacggccgcaaggttgaaactcaaaggaattgacgggggcccgcacaagcggtggatgatgtggattaattcgatgcaacgcgaagaaccttacctacccttgacatgccaggaatcccggaganatctggggg

SEQ ID NO: 7 - DNA, 20 bp, Unknown
  27F
agagtttgatcctggctcag

SEQ ID NO: 8 - DNA, 19 bp, Unknown
  1492R
ggttaccttgttacgactt

SEQ ID NO: 9 - DNA, 17 bp, Unknown
  Rcyc_69F
acgggggcaaccctggt

SEQ ID NO: 10 - DNA, 20 bp, Unknown
  Rcyc_149F
ataacctggcgaaagccagg

SEQ ID NO: 11 - DNA, 20 bp, Unknown
  Rcyc_168R
cctggctttcgccaggttat

SEQ ID NO: 12 - DNA, 21 bp, Unknown
  Rcyc_1446R
ctaccagaagcagttagccta